(19) United States
(12) Patent Application Publication
(10) Pub. No.: US 2005/0053681 A1
(43) Pub. Date: Mar. 10, 2005

(54) Solutions of alkoxylated alkanol amide surfactants and antimicrobial compounds

(75) Inventors: John L. Gormley, Midland Park, NJ (US); James E. Reilly, Wanaque, NJ (US)

Correspondence Address: MAYER, BROWN, ROWE & MAW LLP, 1909 K STREET, N.W., WASHINGTON, DC, 20006, US

(73) Assignee: ICI Americas, Inc., Bridgewater, NJ (US)

(21) Appl. No.: 10/965,721

(22) Filed: Oct. 18, 2004

Related U.S. Application Data
(63) Continuation of application No. 10/161,447, filed on May 30, 2002.
(60) Provisional application No. 60/294,587, filed on Jun. 1, 2001.

(51) Int. Cl. A61K 7/075; A61K 31/075; A61K 35/78

(52) U.S. Cl. 424/769; 514/721

(57) ABSTRACT

A visually clear and substantially colorless antimicrobial-containing solution comprising at least 20 weight percent of an alkoxylated monoalkanolamide surfactant represented by formula II: 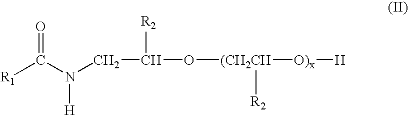 wherein:
R₁ represents a hydrocarbon radical;
R₂ represents a hydrogen atom, —CH₃ or —CH₂—H₃ radical; and x independently represents at least 1. The antimicrobial-containing solutions are suitable for readily mixing into cosmetics and disinfectant cleaning products.

### FIELD OF THE INVENTION

The present invention relates to solutions of alkoxylated alkanolamide surfactants and antimicrobial compounds and to the method of making and using the same. More particularly, the antimicrobial containing solutions of the present invention are liquid at ambient temperatures.

### BACKGROUND OF THE INVENTION

Antimicrobial compounds, particularly halogenated compounds, are typically solids that are processed into powders. Formulators are often attempting to employ such compounds into personal care and detergent products. However, the low water-solubility of these antimicrobial compounds makes working with these compounds challenging. In order to increase solubilization of antimicrobial compounds it has often been necessary to mix these into product formulations in a procedure that, often has required heating the formulation and/or prolonged mixing times. Both of these requirements are undesirable.

Triclosan, 2,4,4′-trichloro-2′-hydroxy-diphenyl ether, is a fairly popular antimicrobial compound. It is available in a solution with propylene glycol as the solubilizer. Although this offers the advantage of providing an antimicrobial in a solution, it still presents shortcomings in that the solution may have lower than desired compatibility with typical liquid formulations where the triclosan can precipitate out if it is added rapidly without sufficient stirring (i.e., special precautions must be adhered to when mixing this solution into product formulations). Furthermore, the solubilizer propylene glycol has several disadvantages that carry over to its use in antimicrobial preparations. These include viscosity reduction in cleansing systems and purported toxicological activity, such that it has been regulated out of cosmetic products in some countries.

### SUMMARY OF THE INVENTION

It is an object of the present invention to use surfactants, alkoxylated alkanolamide compositions, to solubilize antimicrobial compounds and form a solution that can be readily blended into cosmetics, therapeutics and disinfectant products (including, for example, cleansers, hard surface cleaners, cleansing foams, shampoos, body washes, and solid detergents such as powders and bar soaps). In particular, the present invention is directed to visually clear and substantially colorless solutions comprising natural antimicrobial compounds, in particular, and halogenated antimicrobial compounds including halogenated hydroxy-diphenyl ethers such as those represented by Formula I: 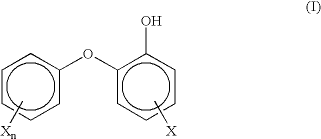
wherein
n is 1 or 2; and
X independently represents a chlorine atom, a bromine atom, or a hydrogen atom, and preferably at least one X represents a chlorine atom, and more preferably at least two X's represent chlorine atoms, and most preferably the antimicrobial compound is triclosan, (2,4,4′-trichloro-2′-hydroxydiphenyl ether).

It is an additional object of this invention to provide a composition in which the antimicrobial compound dissolves in the surfactant under cold-mixing conditions, that is at a temperature of about 15° C. to about 30° C. preferably at an ambient environment of about 18° C. to about 25° C.

It is yet another object of the present invention to provide antimicrobial containing solutions comprising alkoxylated alkanolamide surfactant compositions and antimicrobial compounds, having a melting point of 20° C. or lower.

It is a further object of the present invention to provide methods of preparing antimicrobial containing solutions (including premixtures and cosmetic, therapeutic, and disinfectant products) by first mixing the antimicrobial compound with an alkoxylated alkanolamide.

It is still a further object of the present invention to provide a therapeutic and a method for treatment of Herpes Simplex.

It is yet a further object of the present invention to provide an alkoxylated alkanolamide surfactant composition that provides comparable foam stabilization and viscosity building properties as a surfactant composition comprising a comparable amount of a corresponding alkanolamide (i.e., non-alkoxylated alkanolamide). A further object of this invention is for this alkoxylated alkanolamide surfactant composition to readily dissolve antimicrobial compounds and thus present the antimicrobial compound in a liquid form so that it is easy to handle and readily processible into liquid system, preferably aqueous systems.

These and other objects will become apparent from the description to follow.

### DESCRIPTION OF THE EMBODIMENTS

For purposes of this application, it will be understood that although a particular surfactant compound is named, it refers to a mixture that may comprise additional components such as by-products, unreacted components, and/or catalysts and the like resulting from the formation of the particular surfactant.

The present invention includes forming solutions comprising at least one alkoxylated alkanolamide surfactant composition with at least one antimicrobial compound. A wide variety of alkoxylated alkanolamide surfactants are suitable for forming these solutions. Preferably, the alkoxylated monoalkanolamide surfactant includes those represented by Formula II: 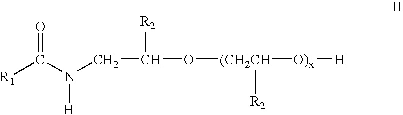
wherein:
R₁ represents a hydrocarbyl radical, preferably an optionally substituted or unsubstituted, branched or straight chain, saturated or unsaturated C₃-C₂₁ hydrocarbyl radical, and more preferably a branched or unbranched C₃-C₂₁ alkyl radical or a mixture thereof;
R₂ independently represents a hydrogen atom, a C₁-C₆ hydrocarbyl radical or a mixture thereof, and preferably a hydrogen atom, C₁-C₂ alkyl or a mixture thereof and more preferably wherein in at least one R₂ is not hydrogen; and
x is an average value of greater than 0.2, and preferably a number representing the number of moles sufficient to provide a surfactant having a melting point of 20° C. or lower.

Suitable alkoxylated alkanolamide surfactants include those discussed in U.S. patent application Ser. No. 09/038,736, filed Mar. 11, 1998 (abandoned), continuation-in part thereof Ser. No. 09/334,812, filed Jun. 17, 1999, and continuation thereof Ser. No. 09/793,042 filed Feb. 26, 2001, the entire disclosures of these are hereby incorporated by reference and Japanese Patent Publication Hei 8-337560 (Kawaken Fine Chemicals Co. Examples of preferred alkoxylated alkanolamide compounds include polyoxypropylene-, polyoxybutylene-, fatty ethanolamides wherein the fatty ethanolamide moiety is derived preferably from lauric monoethanolamide, capric monoethanolamide, capryl monoethanolamide, caprylic/capric monoethanolamide, decanoic monoethanolamide, myristic monoethanolamide, palmitic monoethanolamide, stearic monoethanolamide, isostearic monoethanolamide, isostearic monoisopropanolamide, oleic monoethanolamide, linoleic monoethanolamide, octyldecanoic monoethanolamide, 2-heptylundecanoic monoethanolamide, coconut oil fatty monoethanolamide, beef tallow fatty monoethanolamide, soy oil fatty monoethanolamide and palm kernel oil fatty monoethanolamide. Of these capryl, stearic, isostearic, soy oil, and coconut oil fatty monoethanolamides are particularly preferred.

Additional suitable alkoxylated alkanolamides include alkoxylated monoethanolamide composition mixtures derived from triglyceride fats and oils having the Formula: 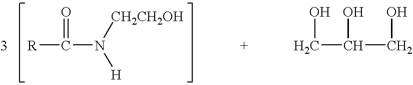
wherein R is the same as R₁ described in Formula II, above. Preferred triglycerides from which the monoethanolamide composition mixtures may be prepared include glyceride esters of acids such as octanoic acid, decanoic acid, lauric acid, myristic acid, palmitic acid, stearic, acid, oleic acid, linoleic acid, linolenic acid, or mixtures thereof as are found in coconut oil, palm oil, sunflower oil, soybean oil, rapeseed oil, castor oil, fish oil, tallow fat, milk fat, lard and other natural sources or may be of synthetic origin. As is known, the solid monoethanolamide composition mixtures suitable for use in the preparation of alkoxylated ethanolamides of the present invention, derived from triglycerides, contain mixtures which are predominantly monoethanolamide derivatives of monoethanolamine, e.g., 3 moles, and small amounts of glycerin, e.g., 1 mole. Such monoethanolamide composition mixtures are typically used as prepared without the need for separation of the glycerin component from the monoethanolamide composition.

Other components that may be present as part of the alkoxylated alkanolamide surfactant component include alkoxylated glycerin, glycerin and non-alkoxylated monoalkanolamide the total amount of which generally ranges from 10% to about 55% by weight. The relative concentration of such additional components depends on the degree of alkoxylation of the reaction mixture and the monoalkanolamide composition mixture from which the modified monoalkanolamide composition mixture of the invention is prepared.

The alkoxylated alkanolamide of the present invention are liquids at ambient temperature (25° C.), and preferably have a melting point temperature lower than 20° C. Preferred alkoxylated ethanolamides include those having a melting point lower than 20° C. and exhibit comparable foam stabilization and viscosity building properties to that of the corresponding monoethanolamides from which it is derived.

In general, the alkoxylated alkanolamide of the present invention may be formed from any suitable method known including reacting the corresponding alkanolamide with a suitable amount of alkylene oxide or mixture of alkylene oxides (including preferably ethylene oxide, propylene oxide, butylene oxide or mixtures thereof) in the presence of a suitable catalyst (such as potassium hydroxide, sodium alcoholate and the like). The degree of alkoxylation of the alkanolamide being treated is important but may be varied depending upon the molecular weight of the alkanolamide and the degree of unsaturation in the fatty alkyl amide moiety. Generally, the alkoxylated alkanolamide is formed by adding at least 0.2 to about 8 moles, preferably from 1 to 4 moles, of ethylene oxide, propylene oxide, butylene oxide or mixtures thereof, per mole of the monoalkanolamide component. Minimum quantities of propylene oxide needed to liquefy some exemplary monoethanolamides at 15° C. are presented in Table 1.

TABLE 1

| | | | Amide |
|---|---|---|---|
| | | Min. weight percent | Molecular |
| | | Propylene Oxide to | Weight - |
| | | Liquefy the | 0.5 |
| Type of | Moles of | Monoethanolamide @ | (Iodine |
| Monoethanolamide | Propoxylation | 15° C. | Value |
| Caprylic/Capric | 1 | 22.82 | 202 |
| Coconut | 2 | 31.73 | 245.5 |
| Soy | 3 | 35.12 | 257 |
| Lard Oil | 4 | 43 | 291 |
| Stearic | 8 | 58.7 | 327 |

Table 2 presents the pour point behavior (° C.) of Caprylic/Capric monoethanolamide with one mole of propoxylation when the indicated amounts of glycerin or glycerin propoxylate is present.

TABLE 2

| | 0% | 5% | 10% | 10% |
|---|---|---|---|---|
| Glycerin | 22.3 | 20.1 | 18.2 | 17.9 |
| Glycerin with 1 mole of | 22.3 | 20.7 | 19.4 | 17.9 |
| propoxylation | | | | |
| Glycerin with 2 moles of | 22.3 | 21.5 | 19.9 | 19.4 |
| propoxylation | | | | |
| Glycerin with 3 moles of | 22.3 | 22.3 | 20.0 | 18.8 |
| propoxylation | | | | |

The alkoxylated alkanolamide surfactant of the present invention generally will contain at least about 60%, preferably about 70%, more preferably about 85% by weight, of the named alkoxylated alkanolamide.

Suitable antimicrobial compounds for forming the solutions of the present invention include natural antimicrobial compounds, in particular, tea tree oil and halogenated hydroxy-diphenyl ethers. More specifically these compounds include halogenated hydroxy-diphenyl ethers represented by Formula I 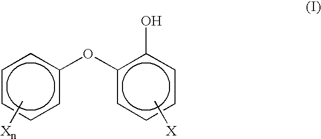
wherein
n is 1 or 2; and
X independently represents a chlorine atom, a bromine atom, or a hydrogen atom, and preferably at least one X represents a chlorine atom, and more preferably at least two X's represent chlorine atoms, and most preferably the antimicrobial compound is triclosan, (2,4,4′-trichloro-2′-hydroxydiphenyl ether).

Halogenated hydroxy-diphenyl ethers are common antimicrobial compounds used in disinfectant cleansing products. They are typically solids at room temperature and require a solvent, heat and/or long mixing times to be brought into an aqueous solution. The alkoxylated alkanolamides discussed herein are able to dissolve the halogenated compounds rapidly, without heat and with only modest stirring (shear).

To prepare the solutions of the present invention, the alkoxylated alkanolamide surfactant will be present in an amount of at least 20 wt. %, preferably at least 50 wt. %. Preferred solutions of the present invention include those where the antimicrobial compounds are mixed with the alkoxylated alkanolamide surfactant in ratios between 5:95-50:50 by weight, and more preferably a ratio between 20:80-33:66 by weight and then mixed by any suitable means by cold-mixing at about 15° C. to about 30° C., preferably at ambient temperature (about 18° C. to about 25° C.

Thus, one of the advantages of the solutions of the present invention is that the solution does minimize or eliminate the need for heating the antimicrobial compound to prepare the solution. Similarly, any heat required to add the solution to a cosmetic or disinfectant cleaning product is minimized or eliminated. This avoids any concerns related to exposing the antimicrobial compound to elevated temperatures. A characteristic of the present invention is that the solution is visually clear and substantially colorless, being at most only slightly tinted and shelf-life stable, for at least 3 months and more preferably at least 6 months and thermally stable, remaining stable at elevated temperatures including 45° C. and higher, and preferably 60° C. and higher. Preferred solutions of the present invention include those that are visually clear and essentially colorless and preferably remain colorless over time and upon exposure to elevated temperatures or after returning to ambient temperatures after sub-ambient exposure. Solubilization is sufficient even if the clear colorless solutions have a slight tint, such as very pale or straw yellow. For instance, the clear solution may have a colour value of about 1 to 3 on the Gardner Colour Value (GSV) scale or it may have a somewhat higher GSV up to 8. Colors with GSV values ranging up to 8 are considered to be light and tints with such values are acceptable in accordance with this invention, with GSV of below 5, and especially below 3, being preferred.

The solution or premixture of the present invention ideally can be readily added to cosmetics and disinfectant cleansing products, including personal care products, household products, industrial cleaners, health care facility cleaners, pharmaceutical production facility cleaners, manufacturing facility cleaners, automotive care products, pet care products, therapeutic products and similar protecting and cleaning products in an overall composition between 0.1-10 wt. %, preferably 0.1-5 wt. % of the premixture, relative to the total weight of the product including the premixture. Preferably, solution is understood to be a solution wherein at least 98 weight % and preferably at least 99.5 weight %, relative to the starting amount of antimicrobial compound in solution, after 2 months and preferably after 3 months of storage at temperatures greater than 45° C. (without agitation).

### EXAMPLES

The following terms are used in the Examples:
AOS-Alpha-olefin sulfonate (40% by weight active aqueous solution);
DI Water—deionized water;
Irgasan® PG60—a liquid which contains 60% triclosan in propylene glycol commercially available from Ciba;
Monoamid® 705—coconut oil diethanolamide commercially available from Uniqema, a business unit of ICI Americas Inc.;
Monateric® CAB—cocamidopropyl betaine (35% solids aqueous solution) commercially available from Uniqema a business unit of ICI Americas Inc.;
Promidium® CC—a propoxylated caprylic/capric monoalkanolamide commercially available from Uniqema, a business unit of ICI Americas Inc.;
Promidium® CO—a propoxylated coconut oil monoalkanolamide commercially available from Uniqema, a business unit of ICI Americas Inc.;
Promidium® SY—a propoxylated soy oil monoalkanolamide commercially available from Uniqema, a business unit of ICI Americas Inc.;
(SLES)—Sodium Lauryl(ethoxy-2)sulphate (28% by weight active aqueous solution);
Triclosan—2,4,4′-trichloro-2′-hydroxy-diphenyl ether commercially available from Sino Lion as Oletron®;
Time for solution to clear—refers to the time (in hours) for the bulk solution to become clear even though there is a significant amount of agglomerated triclosan precipitate dispersed throughout the formulation; and
Dissolution time—refers to the time (in hours) required for the solution to become clear and free of triclosan precipitates.

### Examples 1-12 and Comparative Examples A and B

A series of premixture compositions (or solutions) were prepared and tested for solubility. The premixture compositions were prepared by mixing a surfactant with an antimicrobial compound, triclosan, in the amounts and types set forth in Table 3 below. These surfactants were placed in a 250 ml beaker containing a magnetic stirrer. The triclosan was placed uniformly on top of the surfactant. The mixture was stirred at the lowest setting that created a mild vortex. Each of premixes 1-12 is visually clear and substantially colorless. As set forth in Table 3A, Premixes 1-8 were essentially colorless with GSV's below 5 and generally below 3. Premixes 9-12 revealed light tint with GSV's somewhat below 8

TABLE 3
Surfactant-Triclosan Premixture Compositions

| | Promidium ® | Promidium ® | Promidium ® | Monamid ® | | | |
|---|---|---|---|---|---|---|---|
| | CO | CC | SY | 705 | Triclosan | Weight | Dissolution |
| Example | (weight %) | (weight %) | (weight %) | (weight %) | (weight %) | Ratio* | Time |
| 1 | 100 | | | | 100 | 50:50 | 3 |
| 2 | 134 | | | | 66 | 66:33 | <1 |
| 3 | 160 | | | | 40 | 80:20 | <1 |
| 4 | 190 | | | | 10 | 95:5 | <1 |
| 5 | | 100 | | | 100 | 50:50 | 3 |
| 6 | | 134 | | | 66 | 66:33 | <1 |
| 7 | | 160 | | | 40 | 80:20 | <1 |
| 8 | | 190 | | | 10 | 95:5 | <1 |
| 9 | | | 100 | | 100 | 50:50 | 3 |
| 10 | | | 134 | | 66 | 66:33 | <1 |
| 11 | | | 160 | | 40 | 80:20 | <1 |
| 12 | | | 190 | | 10 | 95:5 | <1 |
| Comp.** A | | | | 100 | 100 | 50:50 | not soluble |
| Comp.** B | | | | 160 | 40 | 80:20 | 1.5 |

Table Note:
*Weight ratio of surfactant to triclosan.
**Comp. indicates a Comparative Example.

TABLE 3A
Surfactant-Triclosan Premixture Compositions
Gardner Colour Value (GSV)

| | Promidium ® | Promidium ® | Promidium ® | Monamid ® | | | |
|---|---|---|---|---|---|---|---|
| | CO | CC | SY | 705 | Triclosan | Weight | |
| Example | (weight %) | (weight %) | (weight %) | (weight %) | (weight %) | Ratio* | GSV |
| 1 | 100 | | | | 100 | 50:50 | 4.7 |
| 2 | 134 | | | | 66 | 66.5:33.5 | 4.1 |
| 3 | 160 | | | | 40 | 80:20 | 2.3 |
| 4 | 190 | | | | 10 | 95:5 | 1.3 |
| 5 | | 100 | | | 100 | 50:50 | 3.2 |
| 6 | | 134 | | | 66 | 66.5:33.5 | 2.6 |
| 7 | | 160 | | | 40 | 80:20 | 1.8 |
| 8 | | 190 | | | 10 | 95:5 | 0.3 |
| 9 | | | 100 | | 100 | 50:50 | 7.9 |
| 10 | | | 134 | | 66 | 66.5:33.5 | Not |
| | | | | | | | determined |
| 11 | | | 160 | | 40 | 80:20 | 7.5 |
| 12 | | | 190 | | 10 | 90:10 | 7.3 |
| Comp.** A | | | | 100 | 100 | 50:50 | not soluble |
| Comp.** B | | | | 160 | 40 | 80:20 | 2.8 |

Table Note:
*Weight ratio of surfactant to triclosan
**Comp. indicates a Comparative Example

Similarly, the visually clear premixes of Promidium® compounds and tea tree oil are observed to be substantially colorless. For example, a premix of 150 parts of Promidium CO and 50 parts of tea tree oil reveals a GSV of 3.1.

### Storage Stability

Promidium® CO/triclosan mixtures were tested for stability. The results were obtained using High Pressure Liquid Chromatography (HPLC) with the compositions reported in Examples 1-4 above are listed in Table 4.

TABLE 4
Storage Stability for Examples 1-4

| | | Room Temperature | | | 45° C. HPLC | | | | |
|---|---|---|---|---|---|---|---|---|---|
| | Weight | HPLC Triclosan | | | Triclosan | | 60° C. HPLC | | |
| | Ratio of | Assay | | | Assay | | Triclosan Assay | | |
| PREMIXTURE | Promidium ® | (weight %) | | | (weight %) | | (weight %) | | |
| Example | CO:Triclosan | 1 Mo. | 3 Mo. | 6 Mo. | 1 Mo. | 3 Mo. | 1 day | 2 wks | 4 wks |
| 1 | 50:50 | 48.4 | 50.6 | 51.5 | 50.2 | 50.3 | 48.9 | 49.1 | 49.3 |
| 2 | 66:33 | 34.1 | 33.3 | 34.3 | 35.3 | 32.9 | 33.9 | 34.6 | 34.2 |
| 3 | 80:20 | 21.3 | 20.3 | 20.6 | 19.8 | 20 | 17.2 | 21.5 | 19.5 |
| 4 | 95:5 | N/A | 4.75 | N/A | N/A | 4.6 | 5.1 | N/A | 5.1 |

### Examples 13-22 and Comparative Examples C-I

A series of liquid cleaners were prepared using the above surfactant/triclosan premixtures along with components set forth in Tables 5A-5C below. Two series of experiments were run at either 50-revolutions per minute (low shear experiment) or 200-revolutions per minute (moderate shear experiment) using a 4 blade paddle mixer.

TABLE 5-A
Mixing triclosan into cleaning solutions (under low shear (50 RPM))

| | Example | | | | | | |
|---|---|---|---|---|---|---|---|
| | 13 | Comp. C | Comp. D | 14 | 15 | Comp. E | Comp. F |
| Ingredients | | | | | | | |
| (weight %) | | | | | | | |
| DI Water | 53.7 | 53.7 | 53.6 | 68.5 | 67.6 | 68.4 | 67.5 |
| SLES | 42.3 | 42.3 | 42.3 | | | | |
| AOS | | | | 27.5 | 21.3 | 27.5 | 21.3 |
| Monateric ® CAB | | | | | 7.1 | | 7.1 |
| Premixture Ex 4 | 4.0 | | | 4.0 | 4.0 | | |
| Promidium ® CO | | 3.8 | 3.8 | | | 3.8 | 3.8 |
| Irgasan PG60 | | | 0.3 | | | 0.3 | 0.3 |
| Triclosan | | 0.2 | | | | | |
| Calculated % | 0.2 | 0.2 | 0.2 | 0.2 | 0.2 | 0.2 | 0.2 |
| Active Triclosan | | | | | | | |
| Dissolution Time | <0.5 | >5 | >5 | <0.5 | <0.5 | >5 | >5 |

TABLE 5-B
Mixing triclosan into cleaning solutions (under normal
shear (200 RPM) at 20° C.)

| | Example | | | | | | |
|---|---|---|---|---|---|---|---|
| | | | | | | | Comp. |
| | 16 | 17 | 18 | 19 | 20 | 21 | Ex. G |
| Ingredients | | | | | | | |
| (weight %) | | | | | | | |
| DI Water | 53.7 | 53.7 | 53.7 | 53.7 | 53.7 | 53.7 | 53.7 |
| SLES | 42.3 | 42.3 | 42.3 | 42.3 | 42.3 | 42.3 | 42.3 |
| Premix. Ex. 4 | 1.0 | | | | | | |
| Premix. Ex. 8 | | 1.0 | | | | | |
| Premix Ex. 12 | | | 1.0 | | | | |
| Premix Ex. 2 | | | | 1.0 | | | |
| Premix Ex. 6 | | | | | 1.0 | | |
| Premix Ex. 10 | | | | | | 1.0 | |
| Irgasan PG-60 | | | | | | | 0.8 |
| Promidium ® CO | 3.0 | | | 3.0 | | | 3.2 |
| Promidium ® CC | | 3.0 | | | 3.0 | | |
| Promidium ® SY | | | 3.0 | | | 3.0 | |
| Calculated % | 0.2 | 0.2 | 0.2 | 0.5 | 0.5 | 0.5 | 0.5 |
| Active triclosan | | | | | | | |
| Time for | 0.25 | 0.1 | 0.25 | 0.25 | 0.1 | 0.25 | 0.3 |
| Solution to Clear | | | | | | | |
| Dissolution Time | 0.6 | 0.1 | 0.6 | 0.9 | 1.0 | 0.75 | >5.0 |

TABLE 5-C
Mixing triclosan into cleaning solutions (under moderate shear
(200 RPM) at 20° C.)

| Ingredients | | | |
|---|---|---|---|
| (Weight %) | Example 22 | Comp. Ex. H | Comp. Ex. I |
| DI Water | 67.6 | 67.6 | 67.6 |
| AOS | 21.3 | 21.3 | 21.3 |
| Monateric ® CAB | 7.1 | 7.1 | 7.1 |
| Premix. Ex. 4 | 1.0 | | |
| Triclosan | | 0.2 | |
| Irgasan ® PG60 | | | 0.33 |
| Promidium ® CO | 3.0 | 4.0 | 4.0 |
| Calculated % active Triclosan | 0.2 | 0.2 | 0.2 |
| Time for Solution to Clear | 0.25 | 6 | 3 |
| Dissolution Time | 0.5 | 8 | 5 |

### Examples 23-26

Premixture 4 (Promidium® CO to triclosan in a weight ratio of 95:5) is added in a soap plodder to a hard texture soap base, Natsoap 3020 (commercially available from Acme Hardesty) under ambient conditions. The mixtures were passed twice through a multi-orifice (⅛″ OD) die and passed once through a 1.5″ compression ring. The premixture was easily incorporated into the soap plodding process such that an acceptable soap base was achieved after two processing cycles, reference Table 6.

TABLE 6
Soap with surfactant-triclosan premixtures

Examples

| | 23 | 24 | 25 | 26 |
|---|---|---|---|---|
| Ingredients | wt. % | wt. % | wt. % | wt. % |
| Natsoap 3020 | 92.5 | 90.0 | 90.0 | 85.0 |
| DI water | 5.0 | 5.0 | 0 | 5.0 |
| Glycerin | 0 | 0 | 5.0 | 5.0 |
| Premixture | 2.5 | 5.0 | 5.0 | 5.0 |
| Example 4 | | | | |

It will be evident from the above that there are other embodiments and methods, which while not expressly described above, are clearly within the scope and spirit of the invention. The description above is therefore intended to be exemplary only and the scope of this invention is to be limited solely by the appended claims.

1-36. (Cancelled).

37. A visually clear and substantially colorless solution comprising:
a) an antimicrobial compound selected for the group consisting of a tea tree oil and a halogenated hydroxy-diphenyl ether; and
b) at least 20 weight percent of the solution, of at least one alkoxylated alkanolamide surfactant represented by formula II: 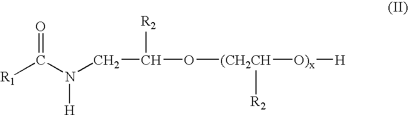
wherein:
R₁ represents a hydrocarbyl radical;
R₂ independently represents a hydrogen atom, a C₁-C₂ alkyl or a mixture thereof, wherein at least one R₂ is not hydrogen; and
x is an average value of greater than 0.2; and
wherein said antimicrobial compound and said at least one alkoxylated alkanolamide surfactant are present in a ratio from between 20:80 to 50:50; and said solution has a Gardner Colour Value (GSV) of below 8.

38. The solution according to claim 37 wherein the antimicrobial compound is selected from tea tree oil or a halogenated hydroxy-diphenyl ether compound represented by Formula I: 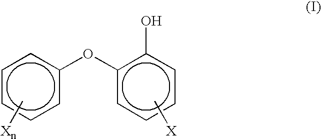
wherein:
n is 1 or 2; and
x independently represents a chlorine atom, a bromine atom, or a hydrogen atom.

39. The solution according to claim 37 wherein the GSV is below 5.

40. The solution according to claim 39 wherein the GSV is below 3.

41. The solution according to claim 38 wherein said antimicrobial compound is said compound represented by Formula I.

42. The solution according to claim 37 wherein the at least one surfactant includes a surfactant represented by formula II: 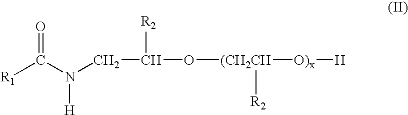
wherein:
R₁ represents a hydrocarbyl radical;
R₂ independently represents a hydrogen atom, a C₁-C₂ alkyl or a mixture thereof, wherein the first R₂ is hydrogen and the second R₂ is C₁-C₂ alkyl; and
x is an average value of greater than 0.2.

43. The solution according to claim 41 wherein the at least one surfactant includes a surfactant represented by Formula II: 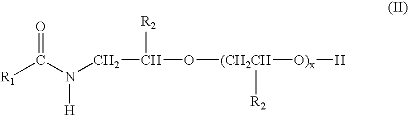
wherein:
R₁ represents a hydrocarbyl radical;
R₂ independently represents a hydrogen atom, a C₁-C₂ alkyl or a mixture thereof, wherein the first R₂ is hydrogen and the second R₂ is C₁-C₂ alkyl; and
x is an average value of greater than 0.2.

44. The solution of claim 43 wherein the solution is a liquid at about 15°-about 30° C.

45. The solution of claim 44 wherein the solution is a liquid at about 18°-25° C.

46. The solution of claim 44 wherein the at least one surfactant is present in an amount of at least 50 weight percent, relative to the total weight of the solution.

47. The solution of claim 44 wherein the at least one surfactant includes an alkoxylated capryl monoalkanolamide, alkoxylated coconut monoalkanolamide, alkoxylated soy oil monoalkanolamide, alkoxylated isostearic monoalkanolamide, alkoxylated stearic monoalkanolamide or mixtures thereof.

48. The solution of claim 37 wherein the ratio of the antimicrobial compound to the at least one surfactant is between 20:80 and 33:66 by weight.

49. The solution of claim 43 wherein at least one X represents a chlorine atom.

50. The solution of claim 43 wherein at least one R₂ is not hydrogen.

51. The solution of claim 43 wherein the antimicrobial compound is triclosan.

52. The solution of claim 43 wherein R₁ is a branched or unbranched C₃-C₂, alkyl radical or a mixture thereof.

53. The solution of claim 43 wherein R₂ independently represents hydrogen atom, or C₁-C₂ alkyl or a mixture thereof.

54. The solution of claim 43 wherein x independently represents a value of from 1 to 4.

55. The solution of claim 47 wherein the at least one surfactant includes a propoxylated capryl monoethanolamide.

56. The solution of claim 47 wherein the at least one surfactant includes a propoxylated coconut monoethanolamide.

57. The solution of claim 47 wherein the at least one surfactant includes a propoxylated soy oil monoethanolamide.

58. The solution of claim 46 wherein the at least one surfactant includes a propoxylated stearic monoethanolamide.

59. The solution of claim 47 wherein the at least one surfactant includes a propoxylated isostearic monoethanolamide.

60. The solution of claim 55 wherein the antimicrobial compound is triclosan.

61. The solution of claim 56 wherein the antimicrobial compound is triclosan.

62. The solution of claim 57 wherein the antimicrobial compound is triclosan.

63. The solution of claim 58 wherein the antimicrobial compound is triclosan.

64. The solution of claim 59 wherein the antimicrobial compound is triclosan.

65. A composition wherein the solution of claim 43 is incorporated into a therapeutic, cosmetic, or disinfectant product.

66. A visually clear and substantially colorless solution comprising:
a) an antimicrobial compound selected for the group consisting of a tea tree oil and a halogenated hydroxy-diphenyl ether; and
b) at least 50 weight percent of the solution, of at least one alkoxylated alkanolamide surfactant represented by formula II: 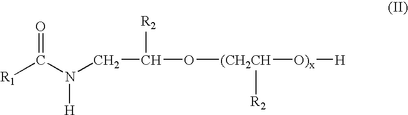
wherein:
R₁ represents a hydrocarbyl radical;
R₂ independently represents a hydrogen atom, a C₁-C₂ alkyl or a mixture thereof, wherein at least one R₂ is not hydrogen; and
x is an average value of greater than 0.2; and
wherein said antimicrobial compound and said at least one alkoxylated alkanolamide surfactant are present in a ratio from between 5:95 to 50:50; and said solution has a Gardner Colour Value (GSV) of below 8.

67. The solution of claim 66 wherein the antimicrobial compound is triclosan.
